(19) United States
(12) Patent Application Publication
(10) Pub. No.: US 2003/0187199 A1
(43) Pub. Date: Oct. 2, 2003

(54) Novel protein and gene

(76) Inventors: Gen Sobue, Aichi (JP); Manabu Douyu, Aichi (JP)

Correspondence Address: GREENBLUM & BERNSTEIN, P.L.C., 1950 ROLAND CLARKE PLACE, RESTON, VA, 20191, US

(21) Appl. No.: 10/149,407

(22) PCT Filed: Dec. 13, 2000
(86) PCT No.: PCT/JP00/08793

(30) Foreign Application Priority Data
Dec. 17, 1999 (JP) 11/359243

(51) Int. Cl. C07K 1/00; C07K 14/00; C07K 17/00

(52) U.S. Cl. 530/350

(57) ABSTRACT

Novel proteins involved in amyotrophic lateral sclerosis and genes encoding the same are provided by a protein having an amino acid sequence shown in SEQ ID NO: 1 or SEQ ID NO: 2, or an amino acid sequence having deletion, substitution or insertion of one or plural amino acids in said amino acid sequences, a gene encoding this protein, an expression vector comprising this gene, a transformant transformed with this expression vector, an antibody against this protein, a method for screening a compound for treatment of a neuro-degenerative disease, a kit for the screening, and a transgenic animal wherein this gene is introduced or deleted.

### TECHNICAL FIELD

The present invention relates to novel proteins and genes thereof. More specifically, the present invention relates to proteins involved in amyotrophic lateral sclerosis, and genes encoding these proteins.

### BACKGROUND ART

Amyotrophic lateral sclerosis (hereinafter referred to as “ALS”) is a neuro-degenerative disease estimated to afflict about 5,000 people in Japan, in which motor neurocytes atrophy and decrease in number, and accompanying this, the muscles controlled by these nerves atrophy. ALS is a disease whose cause is unknown. However, if a factor which specifies the disease is found, it is thought that this factor per se or an inhibitor thereof can be a therapeutic agent against the disease. From this standpoint, identification of this factor is desired, but there is as yet no report that the factor has been cloned.

### DISCLOSURE OF THE INVENTION

The present inventors have purified RNA from the spinal marrow of ALS patients and normal individuals, and have isolated mRNA which specifically increases or decreases in ALS patients by using a molecular indexing. By cloning genes from these mRNA and conducting further examination, the present invention has been completed.

Thus, the present invention provides the following:

(1) A protein having an amino acid sequence shown in SEQ ID NO: 1 or SEQ ID NO: 2, or an amino acid sequence having deletion, substitution or insertion of one or plural amino acids in said amino acid sequences; or a salt thereof.

(2) The protein or salt thereof according to claim 1, which is characterized in that its expression increases in an amyotrophic lateral sclerosis patient.

(3) A gene comprising the nucleotide sequence shown in SEQ ID NO: 3 or SEQ ID NO: 4, or a gene which hybridizes with said gene under stringent conditions and encodes a protein, the expression of which increases in an amyotrophic lateral sclerosis patient.

(4) A gene encoding the protein of claim 1.

(5) An expression vector comprising the gene of claim 3 or 4.

(6) A transformant transformed with the expression vector of claim 5.

(7) A partial peptide of the protein of claim 1 or 2; or a salt thereof.

(8) An antibody against any of the protein of claim 1 or 2 or the partial peptide of claim 7.

(9) A method for screening a compound for treatment of a neuro-degenerative disease, preferably treatment of amyotrophic lateral sclerosis, which is characterized in that the protein of claim 1 or 2 or the partial peptide of claim 7 is used.

(10) A kit for the screening of a compounds for treatment of a neuro-degenerative disease, preferably treatment of amyotrophic lateral sclerosis, which is characterized in that the protein of claim 1 or 2 or the partial peptide of claim 7 is used.

(11) A transgenic animal, wherein the gene of claim 3 or 4 is artificially introduced into a chromosome, or either gene is deleted from a chromosome.

The proteins and genes of the present invention shows an expression or an inhibition of expression, which is specific to ALS patient, and therefore are thought to be involved in the cause and pathology of ALS. Therefore, there is a possibility that the progress of the disease can be halted and the disease can be directed toward improvement by inhibiting or increasing the expression or function of these proteins. That is, these proteins may be a target of therapeutic agents for ALS. Further, since these proteins play ALS patient-specifically, there is a possibility that these proteins become a target protein for easy diagnosis of ALS for which there is no decisive method of diagnosis at present.

Further, the mechanism of onset of most diseases of the neuro-degenerative diseases including ALS is unknown. However, since there is a possibility that they have a common mechanism which finally lead neurocytes to death, there is a possibility that the proteins and genes of the present invention become a target of general therapeutic agents for neuro-degenerative diseases.

### BEST MODE FOR CARRYING OUT THE INVENTION

The present invention will be explained in detail below.

The protein of the present invention is a protein having an amino acid sequence which is identical or substantially identical to the amino acid sequence shown by SEQ ID NO: 1 or SEQ ID NO: 2. The protein having an amino acid sequence which is substantially identical to the amino acid sequence shown by by SEQ ID NO: 1 or SEQ ID NO: 2 is one which has 70% or more, preferably 80% or more, more preferably 90% or more, or most preferably 95% or more identity to the amino acid sequence of SEQ ID NO: 1 or SEQ ID NO: 2, and either which is expressed specifically in ALS patients or the expression of which is inhibited specifically in ALS patients. The expression can be changed depending on the amino acid sequence of the protein. However, to the extent that such expression or inhibition of expression is ALS patient-specific, the amino acid sequence may be changed. Specifically, the protein of the present invention includes fragments or analogs of the proteins having an amino acid sequence which has deletion, substitution or insertion of one or plural, preferably 1 to 10, more preferably 1 to 5, or further preferably, 1, 2, 3 or 4 amino acids in the amino acid sequences of SEQ ID NO: 1 or SEQ ID NO: 2. The fragments and analogs can be obtained by introducing deletion, substitution or insertion of amino acid sequences at a site which is not important for the function of the protein of the present invention. Amino acids at a site which is important for the function of the protein cannot be deleted or substituted. Also, it is not possible to alter sites involved in conservation of the overall structure of the protein in connection with its biological activity, by introducing deletion, substitution or insertion of amino acids. Amino acids which can be substituted are ones having a similar size or polarity. Examples thereof include mutual substitution between aliphatic amino acids, Ala, Val, Leu and Ile, mutual substitution between Ser and Thr which have hydroxyl group, mutual substitution between Asp and Glu which have acidic residue, substitution between Asn and Gln which have amide residue, substitution between Lys and Arg which have basic residue, and substitution between Phe and Tyr which have aromatic residue.

A precursor of the protein of the present invention is also encompassed within the protein of the present invention, so long as it has the above described biological activity. Examples of such precursors include peptides of the present invention to which one or more amino acids are added to the N terminal side and/or C terminal side.

A physiologically acceptable salt of the protein of the present invention or a precursor thereof, is also encompassed by the present invention. Examples of such a salt include acid addition salts including salts of inorganic acids such as hydrochloric acid, phosphoric acid or sulfuric acid, and salts of organic acids such as acetic acid, formic acid, fumaric acid, maleic acid, succinic acid, citric acid, tartaric acid, malic acid, benzoic acid, or benzenesulfonic acid.

The gene of the present invention includes a gene which encodes the above-mentioned protein, and specifically includes a gene having a nucleotide sequence shown by SEQ ID NO: 3, which is a gene encoding a protein having the amino acid sequence shown by SEQ ID NO: 1, or SEQ ID NO: 4, which is a gene encoding a protein having the amino acid sequence shown by SEQ ID NO: 2. Further, a gene which hybridizes under stringent conditions with a gene comprising the nucleotide sequence of SEQ ID NO: 3 or SEQ ID NO: 4, is encompassed within the present invention.

Herein, “a gene which hybridizes under stringent conditions” refers to, for example, a gene which hybridizes with a gene comprising the nucleotide sequence of SEQ ID NO: 3 or SEQ ID NO: 4 under conditions typically used for “probe hybridization.” These genes are cloned by hybridization with the gene of SEQ ID NO: 3 or SEQ ID NO: 4. Specific procedures such as cDNA library preparation, hybridization, positive colony selection, determination of nucleotide sequence, are known, and can be easily performed by reference to, for example, Molecular Cloning 2^{nd} Edt. Cold Spring Harbor Laboratory Press (1989).

In the present invention, a partial peptide refers to a partial peptide composed of at least 6 amino acids among the amino acid sequence of the protein of the present invention. Herein, the limitation of “at least 6 amino acids” is due to the fact that 6 amino acids is the minimum size which can achieve a stable structure. A peptide having a length of 8 amino acids or more is preferred, and a peptide having a length of approximately 10 to 20 amino acids is more preferred. If the partial peptide is a short peptide of about 10 to 20 amino acids, it can be easily synthesized with a peptide synthesizer. If the partial peptide is long, it can be obtained by expressing the peptide prepared according to ordinary gene engineering techniques (for example, by treatment with restriction enzymes, etc.) in animal cells and the like. It is possible to modify a peptide prepared in this manner by ordinary techniques. These partial peptides can be used for the preparation of antibodies as described later.

The protein and gene of the present invention will be explained below.

### 1. Collection of Samples

Spinal cords are collected by surgical methods from the lumbar region of a deceased ALS patient, and also a person having a normal lumbar region who died of a cause other than ALS, in as short a time as possible after death was confirmed.

### 2. Purification of RNA

Purification of RNA from spinal cords can be performed, for example, by using TRIzol TM (GIBCO-BRL) according to the attached protocol.

### 3. Molecular Indexing

Molecular Indexing using the obtained RNA can be performed, for example, according to the method of K. Kato (Nucleic Acids Research 23: 3685 1995).

1) Synthesis of Double Stranded cDNA

Using an oligo dT primer, cDNA is synthesized from RNA with reverse transcriptase. Further, a double stranded cDNA is synthesized using DNA polymerase

2) Cleavage With Class IIS Restriction Enzymes

Next, cDNA is cleaved respectively with three types of class IIS restriction enzyme. For example, Fok II, BsmA I and BsmF I can be used. Class IIS restriction enzymes are restriction enzymes which cleave a site detached by a certain distance from the recognition site, and produce a sticky end having random sequence at the cleaved end. Three groups are produced for each restriction enzyme.

3) Linking of Biotinated Adapter

64 types of biotinated adapter having 3′-end protruding ends corresponding to the 64 permutations of 4-base sticky ends produced in 2) above, are synthesized. Each adapter is linked to cDNA having a matching sequence using E. coli DNA ligase, concentrated with streptavidin coated magnetic beads, and collected. After adapter ligation, cDNA is treated with two types of class IIS restriction enzymes other than the enzymes used in the initial cleavage. Thereby, only cDNA wherein the recognition sites of the initially used class IIS restriction enzyme are most proximate to the poly A tail, is selected. In combination with 2), the cDNA are divided into 192 groups.

4) PCR

After cleaving with restriction enzymes, the double stranded cDNA is alkali denatured using 0.1N NaOH to form single strands. Thereafter, using a primer set of a sense primer which is complementary to the adapter common sequence, and 3 types of antisense anchor oligo dT primer to which are linked A, C or G being complementary to T, G or C as the bases proximate to the poly A tail, PCR is performed for a further division into 3 for each of the adapter-linked groups, to produce 576 types of PCR product.

5) Polyacrylamide Electrophoresis

The 576 types of PCR product obtained in 4) above are run on a denaturing polyacrylamide gel. Fragment analysis is performed using an auto sequencer, and upon comparison of a person having a normal lumbar region and ALS patient, cDNAs are selected which show increased expression that is ALS patient specific.

### 4. Full-Length cDNA Cloning

Using the partial cDNA selected in 3) to 5) as a probe, screening of a commercially available cDNA library is performed, and sequences of the obtained positive colonies are determined by an ordinary method. Further, using mRNA purified with an oligo dT column from RNA obtained in 1, the 5′ end is elongated by 5′ RACE (Rapid Amplification of cDNA Ends) to obtain the beginning of the open reading frame. The sequence of the elongated cDNA is determined by an ordinary method.

Using the thus obtained gene encoding the protein of the present invention, it is possible by gene recombinant techniques to produce the protein in a large scale and use it for medical uses and the like.

That is, a prokaryotic or eukaryotic host cell can be transformed by incorporating the gene encoding the protein of the present invention into a suitable vector. The expression vector may also contain a replication origin, a selective marker, a promoter, an RNA splice site, a polyadenylation signal and the like.

Further, by introducing a suitable promoter or a sequence involved in trait expression into these vectors, the gene can be expressed in the respective host cell. Further, by ligating a gene encoding another polypeptide to a gene of interest to enable expression as a fusion protein, it is possible to simplify purification, increase the amount of expression, and by performing suitable processing in the purification step, excise a protein of interest.

Among hosts to be used in an expression system, examples of prokaryotic organism host cells include E. coli, Bacillus subtilis and the like. Further, among eukaryotic organisms, eukaryotic microorganism host cells include yeast, slime fungus and the like. Insect cells such as Sf9 may be used as a host cell. Further, examples of animal cell-derived host cells include COS cells, CHO cells and the like.

The proteins which was produced by culturing the transformant transformed with the gene encoding the protein of the present invention as mentioned above, can be isolated intracellularly or extracellularly and can be purified. Ordinary methods used for isolating and purifying a protein can be used for isolation and purification of the protein. For example, various types of chromatography, ultrafiltration, salting out, dialysis and the like can be selected and combined as appropriate.

In the present invention, an antibody is an antibody against either the protein of the present invention, or its partial peptide which can constitute an epitope. The antibody can be easily produced by immunizing an animal such as rabbit by normal methods such as the method described in Shinsaibokogakujikken Protocol p 210 Shujunsha (1993); Shin Seikagaku Jikken Koza 1, Tanpakushitu I, p.389-397 (1992), and the like. Antibodies include polyclonal antibodies and monoclonal antibodies, and methods for producing them are known to those skilled in the art. For example, monoclonal antibodies can easily be produced by using the method described in Protein Experimental Methods for Molecular Biological Research Chapter 4, Yodosha (1994). Further, pseudo-humanized antibodies can be obtained according to Japanese Patent Application Laying-Open (Kokai) No. 62-296890. These antibodies can be used as diagnostic agents and experimental reagents used in various immunological assay methods including enzyme immunoassay such as ELISA, radio immunoassay and immunofluorescent methods, and also can be used in affinity chromatography, screening of cDNA library, and the like.

The protein of the present invention can be used in the screening of a medicament for treatment of a neuro-degenerative disease, preferably ALS. That is, under conditions where a candidate compound for the medicament and the protein or partial peptide of the present invention can interact with each other , and in the presence of a second component which can provide a detectable signal depending on the interaction between the candidate compound and the protein or partial peptide of the present invention, the candidate compound and the protein or partial peptide of the present invention are allowed to interact, and by detecting the presence or absence of a signal produced by the interaction, a determination can be made as to whether or not the candidate compound inhibits or activates the activity of the protein or partial peptide of the present invention.

In the present invention, a transgenic animal refers to what is known as a transgenic animal where the gene of the present invention has been artificially introduced into a chromosome of an animal other than a human, and what is known as a knock-out animal where the gene has been deleted from a chromosome thereof. These transgenic animals can be easily produced by those skilled in the art based on known methods such as one regarding a disease model mouse (e.g. Molecular Medicine-Interim Edition, Nakayama Shoten (1994)). The transgenic animal is extremely useful as a model animal for the development of therapeutic agents for a neuro-degenerative disease, preferably, a therapeutic agent for ALS, and as a animal for screening for such therapeutic agents.

### EXAMPLES

The present invention will be explained in more detail below by way of Examples. However, the present invention is not limited by the following Examples so long as the present invention does not exceed the gist. Except where otherwise specified, the procedures in the following Examples were performed according to methods I-V below.

I. Cleavage of DNA with restriction enzymes was carried out with commercially available restriction enzymes using a buffer solution of a composition as indicated on the product, by reacting 2 units of enzyme per 1 μg of DNA at 37° C. for 1 hour.

II. Transformation of E. coli was performed according to the method in Molecular Cloning (1982) p249, Cold Spring Harbor Labs. Purification of plasmids from E. coli was performed by the method described on p86 thereof.

III. Ligation of DNA by T4 DNA ligase was performed as follows. Two fragments to be ligated were dissolved in a ligation buffer [comprising 66 mM Tris-HCl (pH 7.5), 6.6 mM magnesium chloride, and 10 mM dithiothreitol] to 1 μg/10 μl, and 66 μM of ATP was added, and then T4 ligase was added to a concentration of 0.1 units/ μg DNA. Reaction was performed at 4° C. for 18 hours.

IV. For DNA purification from the DNA solution, the following procedures were performed. To a solution containing DNA such as a reaction solution, an equivalent volume of phenol/chloroform was added, and the mixture was mixed vigorously. Then, phenol/chloroform extraction was performed by a centrifugation to collect the aqueous phase containing the nucleic acid.

To the thus obtained aqueous phase, one tenth volume of 3M sodium acetate or an equivalent volume of 4M ammonium acetate, and twice (to the volume of aqueous phase) volume of ethanol were added and mixed. After allowing the mixture to stand overnight at −20° C., or for more than 15 minutes at −80° C., the mixture was centrifuged at 15,000 revolutions, and the nucleic acid was collected as a precipitate, i.e., ethanol precipitation was performed.

V. Excision of DNA from gel was performed by adding elution solution (500 mM NH₄OAc, 10 mM MgCl₂, 1 mM EDTA, 0.1% SDS), reacting at 37° C. for 2 hours, collecting the supernatant, and thereafter performing the method of IV.

### Example 1

1 Collection of Sample

Autopsy was performed within 2 hours after confirmation of death, and the spinal cord was frozen with liquid nitrogen immediately after collection. Only the anterior of the spinal cord was excised with a sterilized scalpel, and purification of total RNA was performed with TRIzolTM (GIBCO-BRL) according to the attached protocol.

2 Molecular Indexing

From 6 μg of the purified total RNA, cDNA was synthesized using an oligo dT primer by using RT-PCR kit ver 2.1 (TAKARA) in accordance with the attached protocol. This cDNA was purified according to IV above, and dissolved in 10 μl of sterilized water. To this was added 1 unit of DNA polymerase I, and double stranded cDNA was synthesized by reacting at 37° C. for 1 hour in a total 20 μl system. This double stranded cDNA was purified according to IV above, and dissolved in 10 μl of sterilized water. Next, cDNA was respectively cleaved with 3 types of class IIS restriction enzyme, Fok II, BsmA I, BsmF I. These were then purified according to IV above and dissolved in 10 μl of sterilized water. Separately, 64 types of biotinated adapter ([Table 1] SEQ ID NO: 5, 6) were synthesized (AMERSHAM-PHARMACIA),

[TABLE 1]!SEQUENCE TABLE (SEQ ID NOS: 5, 6)

| | |
|---|---|
| SEQ ID NO: 5 | 5′ TCAATCGTCATCGATCAGCCC 3′ |
| SEQ ID NO: 6 | 3′ AGTTAGCAGTAGCTAGTCGGGZYXN 5′ |

In SEQ ID NO: 6 of [Table 1], N represents any nucleotide. Z, X and Y each represent four types of nucleotide, and from the combinations which can be adopted, there are 64 types of SEQ ID NO: 6.

Each biotinated adapter and cDNA cleaved with a restriction enzyme were ligated according to the method of III above to obtain 192 types of double stranded cDNA. These were purified according to IV above and were dissolved in 10 μl of sterilized water. Next, 100 μl of streptoavidin coated magnetic beads (GIBCO-BRL) was added, and allowed to react at room temperature for 1 hour. Streptoavidin coated magnetic beads were adsorbed to magnet, and were washed three times with 1 ml of 100 mM Tris-HCl (pH 7.5) to remove non-specifically adsorbed cDNA. Next, cDNA was treated according to I above with two class IIS restriction enzymes other than the enzymes used in the initial cleaving. These were purified according to IV above, and dissolved in 10 μl of sterilized water. 10 μl of 0.2N NaOH was added to the double stranded cDNA, and reacted at room temperature for 10 minutes to form single stranded cDNA. These were purified according to IV above, and dissolved in 10 μl of sterilized water. Using these as a template, the following PCR was performed. Using a primer set of a sense primer (SEQ ID NO: 5) which is complementary to the adapter common sequence labeled with Texas Red, and 3 types of antisense anchor oligo dT primer to which are linked A, C or G being complementary to T, G or C as the base proximate to the poly A tail, PCRs were performed for a further 3 groups for each of the adapter-linked groups.

PCR was performed in a 50 μl system using 1 unit of Taq polymerase (Perkins Elmer), by heating at 94° C. for 5 minutes; performing 30 cycles of 30 seconds at 94° C., 30 seconds at 55° C., and 30 seconds at 72° C.; and then heating at 72° C. for 5 minutes. Thereby, 576 types of PCR product were obtained.

3 Polyacrylamide Electrophoresis

The PCR products obtained in 2 above were run on a 5% denaturing polyacrylamide gel using an auto sequencer (HITACHI). Analysis was performed with SQ5500 (HITACHI), and cDNA having a greatly different expression ratio, BsmFI/39A (133bp) was selected. The selected cDNA was excised from the gel according V above, amplified again by PCR under the same condition, purified with a PCR purification kit (QIAGEN) in accordance with the attached protocol, and then dissolved in 50 μl of sterilized water. 5 μl of this solution was ligated with 50 ng of pT7Blue vector (Novagen) using a Ligation kit (TAKARA) in accordance with the attached protocol. Using this, transformation was performed using a commercially available competent cell, DH5 (TOYOBO) according to the method described in II. According to the method of II, a plasmid was purified from a positive colony and analyzed. Using the vector primer, the cloned gene was analyzed with an auto sequencer (HITACHI).

As a result of examination on the internet using NCBI/BLAST as to gene information of the analyzed sequence, it was found that this sequence was identical to EST, AA21375. Further, from a search of The TIGR Human Gene Index based on this sequence, it was found that this sequence was contained in THC23095.

4 Full-Length cDNA Cloning

Next, using this sequence as a probe, screening of a commercially available human fatal brain cDNA library (TAKARA) was performed according to ordinary methods. An approximately 3.8 kb clone could be obtained from positive colonies. Using the vector primer, the cloned gene was analyzed with an auto sequencer (HITACHI). Further, using 5′ RACE system ver 2.0 (GIBCO BRL), 5′ side elongation reaction was performed in accordance with the attached protocol, thereby elongating it to 4.1 kb. mRNA used at this time, was the product purified using an oligo dT column (Roche) from the initially purified total RNA, 2 μg of which was used. This gene was ligated with pT7Blue vector (Novagen) using Ligation kit (TAKARA). Using this, a commercially available competent cell, DH5 (TOYOBO), was transformed according to II. Using the vector primer, the cloned gene was analyzed with an auto sequencer (HITACHI). As a result of analysis, the gene of SEQ ID NO: 3 was obtained. Further, as a result of translating this sequence, the protein of SEQ ID NO: 1 was obtained.

### Example 2

1 to 3 From Sample Collection to Polyacrylamide Electrophoresis

576 types of PCR product obtained by performing collection of sample and molecular indexing as in Example 1 were similarly run on 5% denaturing polyacrylamide gel, and analysis was performed. BsmFI/58G (54bp) was selected as a cDNA having a greatly different expression ratio. Steps from excision of the selected cDNA from the gel to analysis of the cloned gene, were performed in a manner similar to Example 1. As a result of examination as to gene information of the analyzed sequence, this sequence was found to be identical to EST, AA15380. Further, from a search of The TIGR Human Gene Index based on this sequence, it was found that this sequence was contained in THC213488 (1748bp).

4 Full-Length cDNA Cloning

Screening of a cDNA library as performed in Example 1 was not performed, and using 5′ RACE system ver2.0 (GIBCO BRL), a 5′ side elongation reaction was performed according to the attached protocol, thereby extending DNA by 28 bases to make 1776bp. The mRNA used at this time was the product purified using an oligo dT column (Roche) from the initially purified total RNA, 2 μg of which was used. This gene was ligated with pT7Blue vector (Novagen) using Ligation kit (TAKARA). Using this, a commercially available competent cell, DH5 (TOYOBO), was transformed according to II. Using the vector primer, the cloned gene was analyzed with an auto sequencer (HITACHI). As a result of analysis, the gene of SEQ ID NO: 4 was obtained. Further, as a result of translating this sequence, the protein of SEQ ID NO: 2 was obtained.

### INDUSTRIAL APPLICABILITY

The proteins and genes of the present invention shows an expression or an inhibition of expression, which is specific to ALS patient. Therefore, there is a possibility that the progress of the disease can be halted and the disease can be directed toward improvement by inhibiting or increasing the expression or function of these proteins. That is, these proteins are thought to be useful as therapeutic agents for ALS or in screening thereof. Further, an antibody against the protein of the present invention is thought to be useful in diagnosis of ALS patients.

1. A protein having an amino acid sequence shown in SEQ ID NO: 1 or SEQ ID NO: 2, or an amino acid sequence having deletion, substitution or insertion of one or plural amino acids in said amino acid sequences; or a salt thereof.

2. The protein or salt thereof according to claim 1, which is characterized in that its expression increases in an amyotrophic lateral sclerosis patient.

3. A gene comprising the nucleotide sequence shown in SEQ ID NO: 3 or SEQ ID NO: 4, or a gene which hybridizes with said gene under stringent conditions and encodes a protein, the expression of which increases in an amyotrophic lateral sclerosis patient.

4. A gene encoding the protein of claim 1.

5. An expression vector comprising the gene of claim 3 or 4.

6. A transformant transformed with the expression vector of claim 5.

7. A partial peptide of the protein of claim 1 or 2; or a salt thereof.

8. An antibody against any of the protein of claim 1 or 2 or the partial peptide of claim 7.

9. A method for screening a compound for treatment of a neuro-degenerative disease, which is characterized in that the protein of claim 1 or 2 or the partial peptide of claim 7 is used.

10. The method for screening of claim 9 wherein the neuro-degenerative disease is amyotrophic lateral sclerosis.

11. A kit for the screening of a compounds for treatment of a neuro-degenerative disease, which is characterized in that the protein of claim 1 or 2 or the partial peptide of claim 7 is used.

12. The kit for the screening of claim 11 wherein the neuro-degenerative disease is amyotrophic lateral sclerosis.

13. A transgenic animal, wherein the gene of claim 3 or 4 is artificially introduced into a chromosome, or either gene is deleted from a chromosome.


SEQUENCE LISTING (8 sequences)

SEQ ID NO: 1 - protein, 838 aa, Homo sapiens
MQEQEIGFISKYNEGLCVNTDPVSILTSILDMSLHRQMGSDRDLQSSASSVSLPSVKKAPKKRRISIGSLFRRKKDNKRKSRELNGGVDGIASIESIHSEMCTDKNSIFSTNTSSDNGLTSISKQIGDFIECPLCLLRHSKDRFPDIMTCHHRSCVDCLRQYLRIEISESRVNISCPECTERFNPHDIRLILSDDVLMEKYEEFMLRRWLVADPDCRWCPAPDCGYAVIAFGCASCPKLTCGREGCGTEFCYHCKQIWHPNQTCDAARQERAQSLRLRTIRSSSISYSQESGAAADDIKPCPRCAAYIIKMNDGSCNHMTCAVCGCEFCWLCMKEISDLHYLSPSGCTFWGKKPWSRKKKILWQLGTLVGAPVGIALIAGIAIPAMIIGIPVYVGRKIHNRYEGKDVSKHKRNLAIAGGVTLSVIVSPVVAAVTVGIGVPIMLAYVYGVVPISLCRSGGCGVSAGNGKGVRIEFDDENDINVGGTNTAVDTTSVAEARHNPSIGEGSVGGLTGSLSASGSHMDRIGAIRDNLSETASTMALAGASITGSLSGSAMVNCFNRLEVQADVQKERYSLSGESGTVSLGTVSDNASTKAMAGSILNSYIPLDKEGNSMEVQVDIESKPSKFRHNSGSSSVDDGSATRSHAGGSSSGLPEGKSSATKWSKEATAGKKSKSGKLRKKGNMKINETREDMDAQLLEQQSTNSSEFEAPSLSDSMPSVADSHSSHFSEFSCSDLESMKTSCSHGSSDYHTRFATVNILPEVENDRLENSPHQCSISVVTQTASCSEVSQLNHIAEEHGNNGIKPNVDLYFGDALKETNNNHSHQTMELKVAIQTEI

SEQ ID NO: 2 - protein, 219 aa, Homo sapiens
MEAPGAPPRTLTWEAMEQIRYLHEEFPESWSVPRLAEGFDVSTDVIRRVLKSKFLPTLEQKLKQDQKVLKKAGLAHSLQHLRGSGNTSKLLPAGHSVSGSLLMPGHEASSKDPNHSTALKVIESDTHRTNTPRRRKGRNKEIQDLEESFVPVAAPLGHPRELQKYSSDSESPRGTGSGALPSGQKLEELKAEEPDNFSSKVVQRGREFFDSNGNFLYRI

SEQ ID NO: 3 - DNA, 4357 bp, Homo sapiens
  CDS (318)..(2831)
atagtcaccagaagctggaagagtcaaaggacacattctcccctcaagccccagtgggagcacggcccagctggattttggacttctggcctccagaactagacagggcctcacggtgtcacccagggtggaatacagtggtgtgatcatagctcactgcagcctggaattcctgggctcaagcaaccctgccacctcagccttccaagtagctaggactacagaacatccatgatagcagtcttctgtaaatcgaacttttcaagaattctctgaaggaaccaagtaggatattcttacatcatgacttaatgtgaatgcaagaacaagaaataggttttatctctaaatataatgaagggctgtgtgtaaacactgaccctgtctcaattctaacaagcattttagacatgagtttacatcggcaaatgggttcagatcgagatcttcagtcctctgcttcatctgtgagcttgccttcagtcaaaaaggcacccaaaaaaagaagaatttcaataggctccctgtttcggaggaaaaaagataacaaacgtaaatcaagggagctaaatggcggggtggatggaattgcaagtattgaaagtatacattctgaaatgtgtactgataagaactccattttctctacaaatacctcttctgacaatggattaacttccatcagcaaacaaattggagacttcatagagtgccctttgtgccttttgcggcattctaaagacagatttcctgatataatgacttgtcatcacagatcttgtgtggattgcttacgacaatatttaaggatagaaatctctgaaagcagagttaatattagttgcccagaatgtactgaacggtttaatccccatgatattcgcttgatattaagtgatgatgtcttgatggaaaaatacgaagaatttatgcttagacggtggcttgttgcagatcctgattgtaggtggtgtccagctccagactgtggatatgctgtgatagcatttggatgtgccagctgtccaaaattaacttgtgggcgagagggctgtggaacagagttttgctaccactgtaaacagatttggcaccccaaccagacctgtgatgctgctcgacaagagagagcccagagcttacgtttgagaactatacgttcttcatccattagttatagtcaagagtctggagcagcagctgatgatataaagccatgtccacgatgtgctgcttatataataaagatgaatgatgggagctgcaatcacatgacatgtgctgtttgtggttgtgagttttgttggttgtgtatgaaagaaatctcagatttgcattatctaagtccatcaggatgtactttttgggggaagaaaccctggagccgaaagaagaaaatattgtggcaactgggaacactggttggtgctcctgtcggaatcgctttaatagctggcattgctattcctgcaatgattattggcattcctgtgtatgtgggccgcaagattcacaatcgctatgaaggcaaggatgtttcaaagcacaaacggaatttggccatagcaggtggtgtaacgttgtctgtaatcgtgtctccagtagtagctgcagtgactgtaggtatcggtgttcctattatgttagcttatgtctatggcgtagttccaatttctctttgtcgaagcggaggttgtggagtctcagcaggcaatggaaaaggagttaggattgaatttgatgatgaaaatgatataaatgttggtggaactaacacagctgtagacacaacatcagtagcagaagcaagacacaacccaagcataggggagggaagtgttggtgggctgactggcagtttgagtgcaagtggaagccacatggatcgaataggagccatccgagacaacctgagtgaaacggccagcaccatggcactagctggagccagtataacggggagtctgtcaggaagtgccatggtaaactgttttaacaggttggaagtacaagcagatgtacagaaagaacggtacagtctaagtggagaatctggcacagtcagcttgggaacagttagtgataatgccagcaccaaagcaatggcaggatccattctgaattcctacatcccattggacaaagaaggcaacagtatggaggtgcaagtagatattgagtcaaagccatccaaattcaggcacaacagtggaagcagtagtgtggatgatggcagtgccacccgaagtcatgctggcggttcatccagtggcttgcctgaaggtaaatctagtgccaccaagtggtccaaagaagcaacagcagggaaaaaatcaaaaagtggtaaactgaggaaaaagggtaacatgaagataaatgagacgagagaggacatggatgcacagttgttagaacaacaaagcacgaactcaagtgaatttgaggctccatccctcagtgacagtatgccttctgtagcagattctcactctagtcatttttctgaatttagttgttctgacctagaaagcatgaaaacttcttgtagtcatggttccagtgattatcacacccgctttgctactgttaacattcttcctgaggtagaaaatgaccgtctggaaaattccccacatcagtgtagcatttctgtggttacccaaactgcttcctgttcagaagtttcacagttgaatcatattgctgaagaacatggtaacaatggaataaaacctaatgttgatttatattttggcgatgcactaaaagaaacaaataacaaccactcacatcagacaatggaattaaaagttgcaattcagactgaaatttaggcccataaatgctgcagaataattaccactgtacaaccgtgtttggagctggttgaactacatgtgactacttaagtttcaggttaccagcaaaagccgggtttcattatcataatgcagatacattttctgtgttcagcaaggcattgtgtgtcatgtggatcttagttaccaaactatgaagtgaaggctttaaaagtgcattattttaaggataataaatttgaagagcaaagcatgttttgtgtgtttgccacaaaacattgcttgaagcacatacttagatagaaattggtcttaatttatataatcaatataaaatactaatgcaattctacagcattcaaatgaagaaaacttgaggctttagggataagtggttagtgatattttattgaaaccactaaagagataagtttaaaagaactgcataggttactctcagtatatgatactctgtaacatttctatttatatcggcataaatttcattttttttcttcatatgcaatgtggttatataaagcttaatgcagctcatttgctaccatttggatacttagacactttgagcaagattgtggcagtttttgcacaactttgaaatagaaatacctggtactctatcttgtttattgttgatgccatcttagaggaaaaaatgtaaaggtaagtaattaagcatatgacagcaacaaataagatacataaaactacaaaataaagtcccattaggttataagtattacaaaaaatccacctttctctaaggggaagtttgtaccccattgattcttggtgcctttgggatcgactgggttttaatggcctagttatttgaggattttgctgtgttgttttccatgtcttctctggtcaccttggattatatataaaaatacaggaaatagataaacatgaatgtgattaataatgctgaaaaagtattagcctaccaaagacacactcaggctttagtgaataactttacataacctcagtttttaacacatgcatatcttctccaaccatgaaatcaaagcacggtgcagaacttgtaccaagtacaaaaggtccatgtatgattagcattattttcttttgcttttgtttatggacaatgttcagctgacataagcagaagttggccaaaatactgcctgtactgttaatttcctgtataattcacttaaataaaagcaggttaacctcaatgatagcagttaaaatgttctatcttatgtatttcttttaagtattaccattatggtgctactgagcgttttcttttggtaaaaagaaaaatgccatgggctgcagtcttcttccatcacttttccctaccaggtccattaatatgcttataacactagtgccagttattttatttgataatgcttatggtatttgtatatttgtttgcattccaattttgtttaataatgagtgtgtaaactgcatacgttaaataaatgtaaatactaatgtactgctgcaaaaaaaaaaaaaaaaaaaaaaaaaaa

SEQ ID NO: 4 - protein, 838 aa, Homo sapiens; identical to SEQ ID NO: 1

SEQ ID NO: 5 - DNA, 1780 bp, Homo sapiens
  CDS (676)..(1332)
acttccgctgctrtttcgtagccgactgctgaaggctggtttgcgtcgacatggcggttaccctgagtctcttgctgggcgggcgcgtttgcgccgccgtcactcgctgtgggttcgcgacccggggggtggcgggcccaggccctattggccgggagccggaccccgattccgactgggagccggagaacgggagctgcaggaggtggagaggtaccggcttctccccgggccctcagcttgaagcagggcctcgtgctccggcgctccaggccgcgcccccttggcgccgggtgtcctgccgctgcttgcgcagcggcccttgtttcttcttacccgttgttaggggcgcacgtcaggtgttcagctcccctgggaccactggtcctttcattagtgacgtatttcatcatcagtttagagagttcggcatgcttacaggcagttattgttctaggtgttagctttctgggtgtacggagcagctctaagccggcaacatggcccggttgcccttgcgatcaaagagaagagggctgggcgctccatgatttagcctgaggctcttcaaacatccattctgcttccacgcatggcttctgccattggttctcttcccccagcaccctgaaacgacagaaacaagcaatccgattccagaaaattcggtggcaaatggaggcgcctggtgccccgcccaggaccctgacgtgggaagccatggagcagatacggtatttacatgaggaatttccagagtcctggtcagttcccaggttggctgaaggctttgatgtcagcactgatgtgatccgaagagttttaaaaagcaagtttttacccacattggagcagaagctgaagcaggatcaaaaagtccttaagaaagctgggcttgcccactcgctgcagcacctccggggctctggaaatacctcaaagctgctccctgcaggccactctgtatcaggctctttgcttatgccagggcatgaagcctcatctaaagacccaaatcacagcacagctttgaaagtgatagagtcagacactcacaggacaaatacaccaaggagaaggaagggaagaaataaagaaatccaggacctggaggagagctttgtgcctgttgctgcacccctaggtcatccaagagagctgcagaagtactccagtgattctgagagccccagaggaactggcagtggtgcgttgccaagtggtcagaagctggaggagttgaaggcagaggagccagataacttcagcagcaaagtagtgcagaggggccgagagttctttgacagcaacgggaacttcctgtacagaatttgagtcggggcttggcttatggagatgcctcgtgaaacacagctgggcaagtattaatgtatatggaacagcctggatttctgcatatggataagccaccttggaataggaagaggtgttgagcctggactgtgggaggaaagagctgcgtggatagattcaaacttcctgtggtagtgctcccagtctgacctctgtagaccttcagtactcactcttcttgcttaggctctctgtgtgttgaaagccatcccgtgttgcatgtgttgttacaattttctgtgatacttgcaatttatgtttgagaagaagtgaaaagtttgccttctgacctcatttccttcttgatcagtgaacactaacattttggggacaacttagtcaattggttttccttacaacaaaataaagtaaaatgtagcagtcaaaaaaaaaaaaaaaaaaaaaa

SEQ ID NO: 6 - protein, 219 aa, Homo sapiens; identical to SEQ ID NO: 2

SEQ ID NO: 7 - DNA, 21 bp, Artificial
  Primer
tcaatcgtcatcgatcagccc

SEQ ID NO: 8 - DNA, 25 bp, Artificial
  Primer
nnnngggctgatcgatgacgattga